(19) United States
(12) Patent Application Publication
(10) Pub. No.: US 2025/0064663 A1
(43) Pub. Date: Feb. 27, 2025

(54) ANKLE EXOSKELETON DEVICE

(71) Applicant: THE HONG KONG POLYTECHNIC UNIVERSITY, Hong Kong (CN)

(72) Inventors: Ho Lam HEUNG, Hong Kong (CN); Sheung Mei Shamay NG, Hong Kong (CN); Wai Lung Thomson WONG, Hong Kong (CN)

(21) Appl. No.: 18/726,165

(22) PCT Filed: Nov. 30, 2022
(86) PCT No.: PCT/CN2022/135361

§ 371 Date: Jul. 2, 2024

Related U.S. Application Data
(60) Provisional application No. 63/266,422, filed on Jan. 5, 2022.

(51) Int. Cl. A61H 1/02

(52) U.S. Cl. CPC A61H 1/0266; A61H 2201/0103

(57) ABSTRACT

An ankle exoskeleton device for helping a patient to relearn walking by themselves is disclosed. The ankle exoskeleton device includes a leg brace, an insole, and a rotary actuator. The rotary actuator is pneumatically driven to cause rotation of the insole with respect to the leg brace about a central axis. The rotary actuator comprises first and second elastomeric structure, a rotary shaft rotatable about the central axis to generate an output torque for producing a relative rotatory movement of the insole, and a lever. the rotary shaft is adapted to be moved by controlling two opposing fluid pressures inside the rotary actuator. The first elastomeric structure is extendable to push the lever in a clockwise direction, and the second elastomeric structure is extendable to push the lever in an anticlockwise direction, thereby the first and second elastomeric structures are arranged to control the rotary shaft.

### CROSS-REFERENCE TO RELATED APPLICATIONS

This application claims the benefit of U.S. Provisional Patent Application No. 63/266,422, filed on 5 Jan. 2022, which is incorporated by reference herein in its entirety.

### FIELD OF THE INVENTION

The present invention is generally related to an ankle exoskeleton device. Particularly, the present disclosure is related to a pneumatically driven ankle exoskeleton device having an electrical stimulation system. The ankle exoskeleton device is intended to help a patient, particularly a stroke survivor, to relearn walking by themselves.

### BACKGROUND OF THE INVENTION

Human legs play an important role during normal gait, including shock absorption, stance stabilization, energy conservation, and movement motion. However, mobility and flexibility may deteriorate for the elderly. This problem is particularly significant for patients suffering from a stroke or other terminal diseases. The aging population poses a major challenge to the health care system and the social service, as more countries struggler to support the rising number of elderlies with deteriorated limb mobility or joint flexibility.

One of the medical conditions affecting the independency of the elderly and stroke survivors is the impaired ability to independent walking, which is highly correlated to the health-related quality of life (QOL). The elderly and stroke survivors may not be able to walk for long distances and have poor foot clearance in walking. Therefore, there is a tenancy that stroke survivors become inactive after a stroke. Also, the impaired ability to walk directly causes other problems to health, such as muscle weakness, joint contractures, osteoporosis, pressure ulcer, and obesity. Thus, gait rehabilitation is usually the major priority after a stroke.

Foot drop is a gait abnormality in which the dropping of the forefoot happens due to nerve injuries or neurological disorders, such as stroke or multiple sclerosis. Loss of strength and coordination are induced in the dorsiflexors (muscle groups that lie anterior to the ankle joint, including the Tibialis Anterior, Extensor Digitorum Longus, and Extensor Hallucius Longus) and the plantar flexors (muscle groups that lie posterior to the ankle joint, including Gastrocnemius, Soleus, and Peroneal and Posterior Tibial muscles). Therefore, this leads to the development of an abnormal gait pattern that increases energy expenditure and reduces balance ability during walking. Patients would become more prone to falling, tiredness, or joint limb pain when they are walking outdoors alone.

Weak dorsiflexors affect both the stance and swing phases during gait in the aspects of impeding the performance of foot clearance in the swing phase and generating uncontrolled deceleration in the foot at the initial stance. Steppage-type gait pattern is observed due to the increased knee and hip flexion during the swing phase so that the toe can clear the ground during walking. Weak dorsiflexors also affect the ability to control deceleration of the forefoot shortly after heel strike, which often results in an audible foot slap that impacts stance initialization.

On the other hand, weak plantar flexors primarily affect the stance phase by reducing the stability and propulsive power of the forefoot, particularly during limb support. These can only be compensated by involuntarily reducing walking speed and shortening contralateral step length during gait events. Reduced walking speed results in a corresponding reduction in torque needed for forward progression. A shortened contralateral step is thought to increase stability by limiting anterior movement of the center of pressure with respect to the ankle. Patients may maintain a fast-walking pace by activating the hip flexors more to compensate for weak plantar flexors.

Walking is the most common activity and has been promoted as a method for improving both physical and cognitive functions, assisting with the recovery of independence, and preventing physical disability in older adults. Ankle foot orthoses (also referred to as “orthoses” or “AFOs”) can be used to reduce the impacts of lower limb neuromuscular motor function impairments to gait. AFOs can be used as rehabilitation or diagnostic devices, for example, to assist with walking, to directly measure joint range-of-motion or force, or to perturb gait. Existing technologies for AFOs include active or passive devices. Passive devices generally immobilize the ankle into a neutral position (i.e., 90 degrees between the shank and the foot) for the prevention of foot drop. However, passive AFOs produce an unnatural gait and cannot adapt to ongoing walking conditions by providing changing assistance directly during the propulsive phase of gait, for example, push-off. This would dramatically inhibit joint motion at undesirable times.

Existing active AFOs adopt different actuation mechanisms to assist ankle movement. Some active devices or semi-active devices are installed with a spring (linear or torsional) at the articulated joint. The devices are further secured to the ankle for providing assistive force to weak dorsiflexion and braking, but no extra assistance can be provided to aid with propulsion. Examples of semi-active devices include U.S. Pat. No. 2,516,872A “Ankle brace”, U.S. Pat. No. 3,064,644A “Lower leg brace”, U.S. Pat. No. 6,171,272B1 “Short leg brace”, U.S. Pat. No. 6,350,246B1“Ankle and foot therapeutic device”, and U.S. Pat. No. 8,382,694 “Ankle-foot orthotic for treatment of foot drop”. This would also resist joint motion at undesirable times and cannot well assist with gait, which may not be ideal for treating many gait impairments.

Majorities of active AFOs consist of actuators that are mainly controlled by electricity or fluidic pressure for providing direct assistive torque to move the ankle joint. They can mainly aid in propulsive movements necessary for normal gait and clearing the ground. Examples of active devices include U.S. Pat. No. 8,808,214 “Active ankle foot orthosis”, U.S. Pat. No. 8,771,211 “Ankle orthosis” (electrically driven), U.S. Pat. No. 9,480,618B2 “Portable active pneumatically powered ankle-foot orthosis”, and US patent application no. 2019/0336315 A1 “Soft dynamic ankle-foot orthosis exosuit for gait assistance with foot drop” (pneumatic driven). These exoskeleton robots can be programmed to moderate drop foot walking depending on the on-going gait phase, but they are hard to be fit into any footwear without modification. Particularly, active devices have been developed for laboratory settings. They are tethered to giant power sources, and therefore they become cumbersome and unattractive to be used outside the clinic or laboratory. Therefore, active AFOs of the prior art still have issues associated with effectiveness, complexity, speed of operation, and safety.

Accordingly, there is a need in the art to have an ankle exoskeleton device that can effectively support the ankle joint of a patient and stimulate the muscles of the lower limb. Furthermore, other desirable features and characteristics will become apparent from the subsequent detailed description and the appended claims, taken in conjunction with the accompanying drawings and this background of the disclosure.

### SUMMARY OF THE INVENTION

Provided herein is an ankle exoskeleton device for supporting the ankle joint and stimulating the muscles of the lower limb. It is an objective of the present disclosure to help a patient, particularly a stroke survivor, to relearn walking by themselves.

According to the first aspect of the present disclosure, a rotary actuator for use in a robotic device is provided. The rotary actuator is pneumatically driven to cause rotation of a first element with respect to a second element about a central axis. The rotary actuator includes a first elastomeric structure; a second elastomeric structure; a rotary shaft rotatable about the central axis to generate an output torque for producing a relative rotatory movement of the first element; and a lever protruding from the rotary shaft perpendicularly for rotating the rotary shaft. The rotary shaft is adapted to be moved by controlling two opposing fluid pressures inside the rotary actuator. The first elastomeric structure is extendable to push the lever in a clockwise direction, and the second elastomeric structure is extendable to push the lever in an anticlockwise direction, thereby the first and second elastomeric structures are arranged to control the rotary shaft.

According to a further embodiment of the present disclosure, the rotary actuator is coupled to the first element and the second element, wherein the first element comprises an insole, and the second element comprises a leg brace. The insole is pivotally coupled to the leg brace at or proximal to an ankle joint. The insole is movable by the rotary actuator with a range of motion to support the ankle joint to move between plantar flexion and dorsiflexion in a sagittal plane.

According to a further embodiment of the present disclosure, the rotary actuator includes a first inflatable chamber and a second inflatable chamber. The first inflatable chamber is in contact with the first elastomeric structure, and the second inflatable chamber is in contact with the second elastomeric structure. The first and second inflatable chambers are pressurized to provide the two opposing fluid pressures for fixing the lever at a position corresponding to an angle of the first element with respect to the second element.

Preferably, the first and second inflatable chambers are pressurized to provide the two opposing fluid pressures for fixing the lever at the position corresponding to the angle of the first element with respect to the second element.

Preferably, the rotary actuator is provided within a hollow cylinder defining an internal space between an inner wall of the hollow cylinder and the rotary shaft, and wherein the first and second elastomeric structures and the first and second inflatable chambers are accommodated within the internal space.

According to a further embodiment of the present disclosure, the first inflatable chamber receives pressurized fluid at a first pressure from a pressure source; and the second inflatable chamber receive pressurized fluid at a second pressure from the pressure source.

Preferably, the first pressure and the second pressure supplied to the first and second inflatable chambers are controlled by a control valve.

According to a further embodiment of the present disclosure, each of the first and second elastomeric structures comprises undulated peripheral surfaces defining an inner sinusoidal edge and an outer sinusoidal edge; wherein the inner and outer sinusoidal edges allow compression or expansion of the first and second elastomeric structures.

Preferably, the first and second elastomeric structures comprise a structural mesh connecting the inner sinusoidal edge and the outer sinusoidal edge for supporting the first and second elastomeric structures and restricting an expansion of the first and second elastomeric structures in a vertical direction.

Preferably, the structural mesh connects inner peaks of the inner sinusoidal edge with outer peaks of the outer sinusoidal edge; and inner valleys of the inner sinusoidal edge with outer valleys of the outer sinusoidal edge.

Preferably, the structural mesh is formed using supporting lengthwise filaments, supporting widthwise filaments, or a patterned mesh selected from a net mesh or a double helical mesh.

Preferably, the structural mesh is impregnated within the first and second elastomeric structures or mounted to a surface of the first and second elastomeric structures.

According to the second aspect of the present disclosure, an ankle exoskeleton device includes a leg brace; an insole pivotally coupled to the leg brace at or proximal to an ankle joint; a rotary actuator coupled to the leg brace and the insole, the rotary actuator being driven to cause rotation of the insole with respect to the leg brace to support the ankle joint; and an electrical stimulation system comprising a pulse generator and a plurality of electrodes for intermittently stimulating muscles in a lower limb to facilitate a walking gait. The plurality of electrodes are arranged for contacting a plurality of regions of the lower limb to apply electrical current pulses to dorsiflexor muscle group and plantar flexor muscle group. The pulse generator is configured to generate the electrical current pulses of a predetermined current amplitude independent of a voltage level and a human skin resistance across any two of the plurality of electrodes.

According to a further embodiment of the present disclosure, the pulse generator is a closed-loop system comprising a current-controlled source and a transformer having a primary coil connected to the current-controlled source and a secondary coil connected to the plurality of electrodes.

Preferably, the closed-loop system further includes a voltage buffer configured to sense a feedback signal from the plurality of electrodes; a first non-inverting amplifier connected to the voltage buffer to amplify the feedback signal with a gain equal to or larger than 1; and a differential amplifier configured to determine a difference between the feedback signal from the first non-inverting amplifier and an input signal from a characteristic control module.

According to a further embodiment of the present disclosure, the electrical current pulses have a square waveform or a triangular waveform.

According to a further embodiment of the present disclosure, two of the plurality of electrodes are arranged to stimulate the dorsiflexor muscle group, including tibialis anterior muscle; and another two of the plurality of electrodes are arranged to stimulate the plantar flexor muscle group, including soleus, gastrocnemius, and plantaris.

Preferably, the electrical stimulation system is configured to stimulate the dorsiflexor muscle group, and the rotary actuator is configured to support the ankle joint to move in the dorsiflexion during toe-off, mid-swing, terminal swing, and heel strike; and the electrical stimulation system is configured to stimulate the plantar flexor muscle group, and the rotary actuator is configured to support the ankle joint to move in the plantar flexion during terminal stance and pre-swing.

According to a further embodiment of the present disclosure, the ankle exoskeleton device further includes a sensor system for providing kinetic feedback and kinematic feedback. The sensor system includes a first motion sensor placed on the leg brace to sense a change in displacement and orientation of the leg brace; a second motion sensor placed inside the rotary actuator to sense a change in displacement and orientation of the insole; and at least two force sensors placed on the insole at a heel region and a toe region.

Preferably, the ankle exoskeleton device further includes a microcontroller configured to process the readings obtained from the sensor system; control the rotary actuator to cause rotation of the insole with respect to the leg brace; and control the electrical stimulation system to activate electrical stimulation on muscles of the lower limb.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects and advantages of the present invention are disclosed as illustrated by the embodiments hereinafter.

### BRIEF DESCRIPTION OF THE DRAWINGS

The appended drawings contain figures to further illustrate and clarify the above and other aspects, advantages, and features of the present disclosure. It will be appreciated that these drawings depict only certain embodiments of the present disclosure and are not intended to limit its scope. It will also be appreciated that these drawings are illustrated for simplicity and clarity and have not necessarily been depicted to scale. The present disclosure will now be described and explained with additional specificity and detail through the use of the accompanying drawings in which:

FIG. 1 shows the ankle of the user placed inside an ankle exoskeleton device at a standing position in accordance with certain embodiments of the present disclosure;

FIG. 2 is a perspective view of the ankle exoskeleton device without the electrical stimulation system in accordance with certain embodiments of the present disclosure;

FIG. 3A is a side view of FIG. 2 in accordance with one embodiment of the present disclosure;

FIG. 3B is a side view of FIG. 2 during ankle plantar flexion in accordance with one embodiment of the present disclosure;

FIG. 3C is a side view of FIG. 2 during ankle dorsiflexion in accordance with one embodiment of the present disclosure;

FIG. 4A is a side view of the rotary actuator of the ankle exoskeleton device in accordance with another embodiment of the present disclosure;

FIG. 4B is a side view of the rotary actuator during ankle plantar flexion in accordance with another embodiment of the present disclosure;

FIG. 4C is a side view of the rotary actuator during ankle dorsiflexion in accordance with another embodiment of the present disclosure;

FIG. 5 shows a dropped foot;

FIG. 6 shows the electrical stimulation system with a plurality of electrodes for applying electrical current pulses to dorsiflexor and plantar flexor muscle groups in accordance with certain embodiments of the present disclosure;

FIG. 7 is a schematic diagram illustrating the functioning of electrical stimulation system in accordance with certain embodiments of the present disclosure;

FIG. 8 is a graph illustrating an example of multiple footsteps;

FIG. 9 is a graph illustrating an example of force sensor values at a gait phase in accordance with certain embodiments of the present disclosure;

FIG. 10 is a table illustrating an example method for determining muscle stimulation and for controlling the ankle exoskeleton device at different gait events in accordance with certain embodiments of the present disclosure;

FIG. 11 is a flowchart illustrating the operation mechanism of the ankle exoskeleton device in accordance with certain embodiments of the present disclosure;

FIG. 12 is a block diagram of an exemplary ankle exoskeleton device in accordance with certain embodiments of the present disclosure; and

FIG. 13 is an exemplary control interface for controlling the ankle exoskeleton device in accordance with certain embodiments of the present disclosure.

### DETAILED DESCRIPTION OF THE INVENTION

The following detailed description is merely exemplary in nature and is not intended to limit the disclosure or its application and/or uses. It should be appreciated that a vast number of variations exist. The detailed description will enable those of ordinary skilled in the art to implement an exemplary embodiment of the present disclosure without undue experimentation, and it is understood that various changes or modifications may be made in the function and structure described in the exemplary embodiment without departing from the scope of the present disclosure as set forth in the appended claims.

The benefits, advantages, solutions to problems, and any element(s) that may cause any benefit, advantage, or solution to occur or become more pronounced are not to be construed as a critical, required, or essential features or elements of any or all of the claims. The invention is defined solely by the appended claims including any amendments made during the pendency of this application and all equivalents of those claims as issued.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The terms “comprising,” “having,” and “including” or any other variation thereof, are to be construed as open-ended terms (i.e., meaning “including, but not limited to,”) unless otherwise noted. The use of any and all examples, or exemplary language (e.g., “such as”) provided herein, is intended merely to illuminate the invention better and does not pose a limitation on the scope of the invention unless otherwise claimed. No language in the specification should be construed as indicating any non-claimed element as essential to the practice of the invention. Further, unless expressly stated to the contrary, “or” refers to an inclusive “or” and not to an exclusive “or”. For example, a condition A or B is satisfied by any one of the following: A is true and B is false, A is false and B is true, and both A and B are true. Terms of approximation, such as “about”, “generally”, “approximately”, and “substantially” include values within ten percent greater or less than the stated value.

Unless otherwise defined, all terms (including technical and scientific terms) used in the embodiments of the present invention have the same meaning as commonly understood by an ordinary skilled person in the art to which the present invention belongs.

The term “plantar flexion” refers to an extension movement of an ankle such that the foot rotates downwards or inferiorly. The term “dorsiflexion” refers to a flexion movement of an ankle such that the foot rotates upwards or superiorly.

The term “exoskeleton”, as used herein, refers to a mechanical structure with elements coupled in series, which is adapted to be attached to a user's body.

The term “insole”, as used herein, refers to a broader array of footwear products than mere insoles, and those products include orthotics, midsoles, sandals, inserts, and other similar items that provide support to the user's foot.

Embodiments of the present invention include instruments, systems, techniques, and methods for assisted ankle movement. More specifically, the present disclosure may be applied to, but not limited to, robotic ankles, including exoskeleton type robotic ankles. Embodiments can include ankle exoskeleton devices or robotic ankle devices having rotary actuators with electrical stimulation components. In certain embodiments, the present disclosure can be applied in the medical field or for physiotherapy training. In particular, the device disclosed herein helps a patient to relearn walking after stroke, cerebral ischemia, or other cardiovascular diseases, or helps a patient with lower limb gait deficiencies, including, but not limited to, trauma, incomplete spinal cord injuries, multiple sclerosis, muscular dystrophies, or cerebral palsy to walk. Therefore, the purpose of the present disclosure is to provide a device that can be used by the patient for recovering the ankle function.

The present disclosure provides an ankle exoskeleton device that combines the features of soft robotics into a mechanical structure for facilitating the movement of the lower limb. The movement can be adaptive to human gait patterns. In certain embodiments, the ankle exoskeleton device includes an electrical stimulation system for stimulating muscles in a lower limb constantly to strengthen muscle contraction during gait.

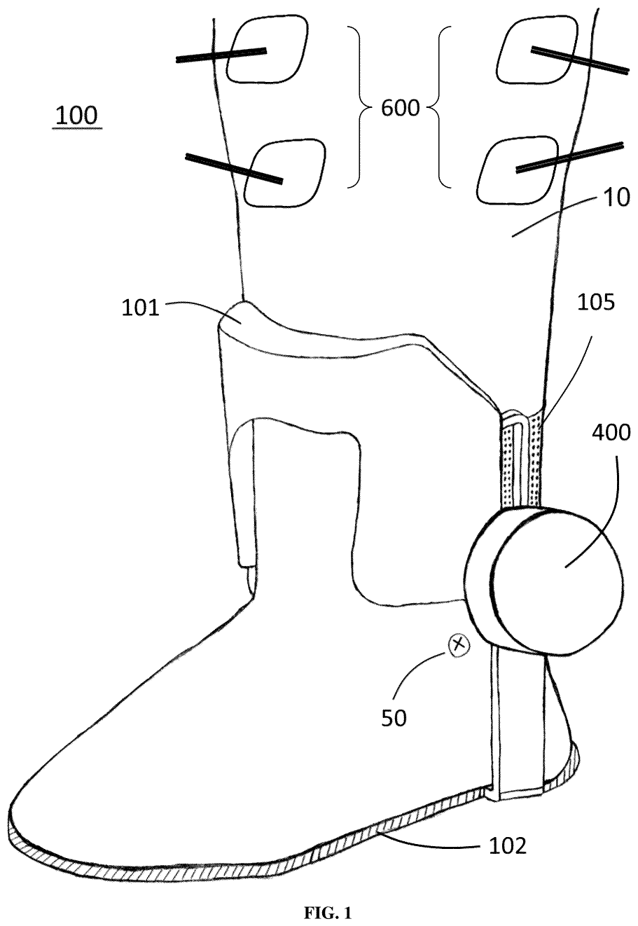

FIG. 1 shows an ankle exoskeleton device 100 in accordance with a first embodiment of the present disclosure. The ankle exoskeleton device 100 may have a lightweight and compact structure, which can be mounted on a user's impaired lower limb. The user may wear the ankle exoskeleton device 100 like a shoe or other rather than a bulky lab-based device. Therefore, the ankle exoskeleton device 100 may be used as a daily assistive device (instead of a rehabilitation device) helping a patient to relearn walking by themselves after the training for facilitating the recovery of ankle function of the patient.

In the illustrated embodiment, the ankle exoskeleton device 100 comprises a leg brace 101, an insole 102 pivotally coupled to the leg brace 101 at or proximal to an ankle joint 50, and a rotary actuator 400 coupled to the leg brace 101 and the insole 102 as a means for securing the rotary actuator 400 to the ankle joint 50. The ankle exoskeleton device 100 may further comprise an electrical stimulation system 600 for intermittently stimulating muscles in a lower limb 10 to facilitate a better walking gait, which may be separated from the leg brace 101 or formed integrally. The leg brace 101 may be secured on the lower limb 10 using fastening means 105. In certain embodiments, the fastening means 105 may be any kind of connection including, but not limited to, Velcro fasteners, Ladder straps, hook and loop connections, buttons, magnetic connectors, zippers, or other similar devices.

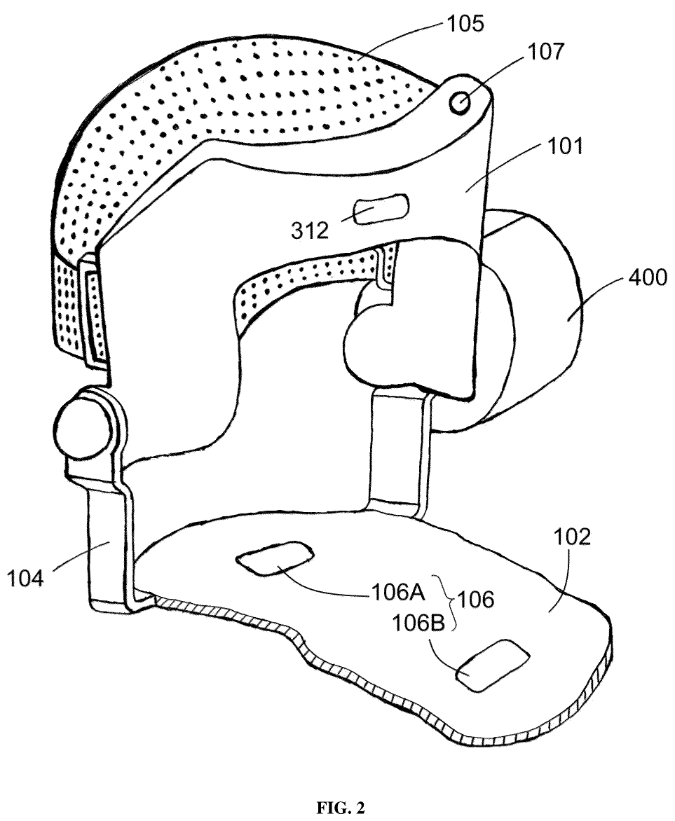

Referring to FIG. 2, the insole 102 may be configured to receive and support at least a portion of the foot. The insole 102 may be fitted into any kind of footwear without occlusion or, if it is an inherent part of the footwear, may replace any part of the unmodified footwear. On the rear side of the insole 102, one or more proximal extensions 104 may be provided to form a physical connection up to the lateral malleolus of the user for coupling the rotary actuator 400. The leg brace 101 and the insole 102 may be padded with soft materials, e.g., rubber, plastic, or silicone, to increase the level of comfort and absorb shock during walking. Similar compliance between the soft materials and human tissues to bring minimal harm to the user. The height of the proximal extension 104 and the width between two proximal extensions 104 may be adjusted to fit one's foot size.

Preferably, the leg brace 101 complies with IPX2 Rainfall Drip Testing. The leg brace 101 and the insole 102 may be made of a biocompatible material since they are directly in contact with the human skin. In particular, the material complies with biocompatibility tests of at least the International Organization for Standardization (ISO) 10993-1 (Evaluation and testing within a risk management process), the ISO 10993-5 (Test for in vitro cytotoxicity), and the ISO 10993-10 (Tests for irritation and skin sensitization).

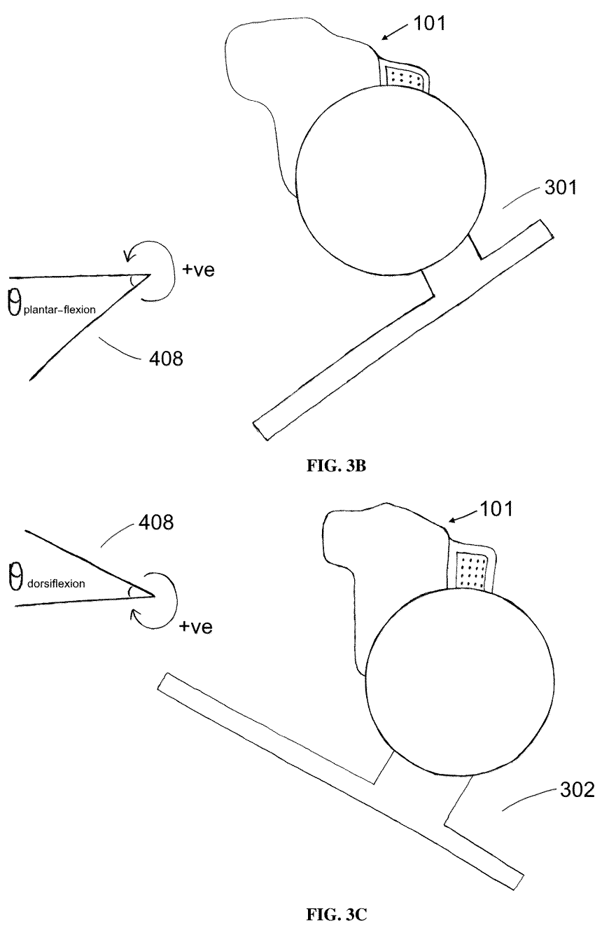

The ankle exoskeleton device 100 may comprise a sensor system that includes one or more sensors integrated into the ankle exoskeleton device 100 to provide kinetic feedback and kinematic feedback about the gait pattern of the user. In certain embodiments, the sensor system comprises a first motion sensor 312 placed on the leg brace 101 to sense the change in displacement and orientation of the leg brace 101, a second motion sensor 311 (shown in FIG. 4A) placed inside the rotary actuator 400 to sense the change in displacement and orientation of insole 102, and a plurality of force sensors placed on the insole 102. In the illustrated embodiment, a first force sensor 106A is installed at the heel region of the insole 102, and a second force sensor 106B is installed at the toe region of the insole 102. The two force sensors 106 may be a thin film force sensitive resistor (FSR), a force transducer, a strain gauge, a pressure sensor, or the like.

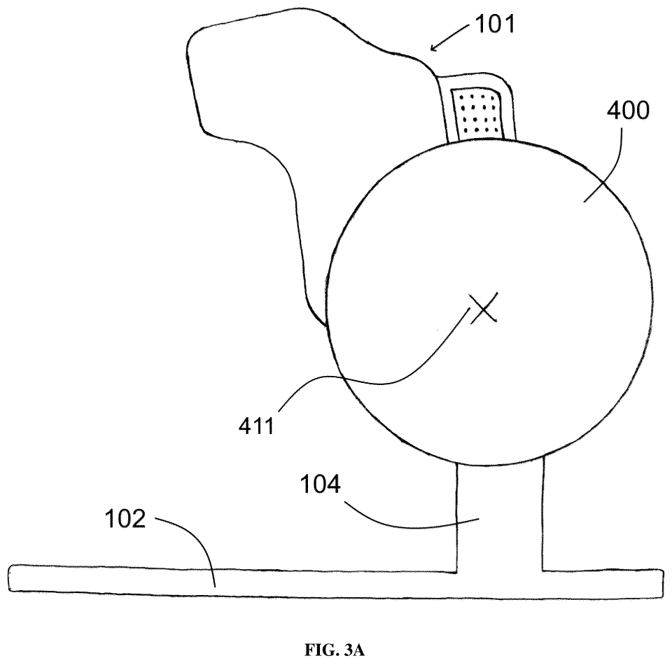

FIGS. 3A-3C show the side view of the ankle exoskeleton device 100 (without the electrical stimulation system 600) for illustrating the configuration for plantar flexion and the dorsiflexion. As the rotary actuator 400 couples to the leg brace 101 and the insole 102, the rotary actuator 400 is driven to cause rotation of the insole 102 with respect to the leg brace 101 about a central axis 411. Advantageously, the ankle exoskeleton device 100 provides an output torque for producing a relative rotatory movement of the insole 102 with a range of motion for moving the ankle joint 50 between plantar flexion 301 and dorsiflexion 302 in the sagittal plane. Refer to the scientific journal article by Blanchette et al. (“Modifications in ankle dorsiflexor activation by applying a torque perturbation during walking in persons post-stroke: a case series”, hereinafter referred to as “Blanchette”), the flexion torque that was necessary to move the ankle joints of six recruited stroke subjects were disclosed in Table 3 of Blanchette. The peak flexion torque value in the ankle during walking ranges from 1.9±0.5 N-m to 4.7±0.6 N-m, as shown in Table I below.

TABLE I
Table of the peak flexion torque values of ankle
during a walking task of 6 participants.

| | Peak Flexion Torque Value in |
|---|---|
| Participant | Ankle during Walking (N-m) |
| S1 | 1.9 ± 0.5 |
| S2 | 3.0 ± 0.6 |
| S3 | 3.2 ± 0.8 |
| S4 | 4.7 ± 0.6 |
| S5 | 2.5 ± 0.5 |
| S6 | 3.0 ± 0.4 |

With reference to the above peak flexion torque, the minimum output torque generated by the rotary actuator 400 in order to provide sufficient support for moving the ankle joint 50 between plantar flexion 301 and dorsiflexion 302 in the sagittal plane can be determined. In one embodiment, the rotary actuator 400 provides an output torque with a range between 1 N-m and 5 N-m.

Alternatively, several studies conducted by Brockett et al., Grimston et al., and Stauffer et al. (“Biomechanics of the ankle”, 2016; “Differences in ankle joint complex range of motion as a function of age”, 1993; “Force and motion analysis of the normal, diseased, and prosthetic ankle joint”, 1977) indicated an overall range-of-motion of the ankle joint in the sagittal plane of between 65 and 75°, moving from 10 to 20° of dorsiflexion through to 40-55° of plantarflexion. Therefore, the rotation 408 of the insole 102 with respect to the leg brace 101 can also be determined.

The first embodiment of the present disclosure provides a rotary actuator 400 coupled to the leg brace 101 and the insole 102 for supporting the foot of the user. FIG. 4A is a side view of the rotary actuator 400 of the ankle exoskeleton device 100 in accordance with another embodiment of the present disclosure. The rotary actuator 400 is pneumatically driven and has a hollow cylinder 401, a rotary shaft 402, and elastomeric structures 403. The hollow cylinder 401 defines an internal space 407 between an inner wall 401A of the hollow cylinder 401 and the rotary shaft 402. The rotary shaft 402 is rotatable about the central axis 411 to generate an output torque for producing a relative rotatory movement of the insole 102 with respect to the leg brace 101. The elastomeric structures 403 include at least a first elastomeric structure 403A and a second elastomeric structure 403B. The elastomeric structures 403 have one end fixed at a position 406 and another end adjustably engaged with a lever 410. The lever 410 is protruded from the rotary shaft 402 perpendicularly for rotating the rotary shaft 402, wherein the first elastomeric structure 403A engages the lever 410 from one side, and the second elastomeric structure 403B engages the lever 410 from another side. The first motion sensor 311 may be mounted on or integrated into the lever 410. The elastomeric structures 403 are arranged to provide two opposing forces on the lever 410 for causing a rotation of the rotary shaft 402. In certain embodiments, the first elastomeric structure 403A is extendable to push the lever 410 in a clockwise direction, and the second elastomeric structure 403B is extendable to push the lever 410 in an anticlockwise direction, thereby the first and second elastomeric structures 403A, 403B are arranged to control the rotary shaft 402 to rotate about the central axis 411.

In certain embodiments, the rotary actuator 400 further comprises a first inflatable chamber 404A and a second inflatable chamber 404B. The first inflatable chamber 404A is in contact with and stacked below the first elastomeric structure 403A, and the second inflatable chamber 404B is in contact with and stacked below the second elastomeric structure 403B. Inside the hollow cylinder 401, the first and second elastomeric structures 403A, 403B and the first and second inflatable chambers 404A, 404B are accommodated within the internal space 407. In one embodiment, the first elastomeric structure 403A and the first inflatable chamber 404A are placed on one side of the lever 410, while the second elastomeric structure 403B and the second inflatable chamber 404B are placed on another side of the lever 410. When the pressure inside the first or second inflatable chambers 404A, 404B increases by supplying pressurized fluid, it will occupy more space and exerts an upward force on the corresponding first or second elastomeric structures 403A, 403B. As the first and second elastomeric structures 403A, 403B are shaped to convert a vertical force to a lateral force, the upward force resulting from the supply of the pressurized fluid will cause the elastomeric structures 403 to expand laterally to push the lever 410 in either a clockwise or an anticlockwise direction. As a result, the first and second inflatable chambers 404A, 404B are pressurized to provide two opposing fluid pressures for fixing the lever 410 at a position corresponding to an angle of the insole 102 with respect to the leg brace 101.

Although the illustrated embodiment provides that the rotary actuator 400 is used in an ankle exoskeleton device 100 for moving the insole 102 from the leg brace 101 for supporting the foot of the user, it is apparent that the rotary actuator 400 may also be used in any robotic devices without departing from the scope and spirit of the present disclosure. When the rotary actuator 400 is used in a robotic device, the rotary actuator 400 is pneumatically driven to cause rotation of a first element with respect to a second element about a central axis. The first element may comprise the insole 102, the proximal extension 104, and other connectors. The second clement may comprise the leg brace 101, the fastening means 105, other fluid control components, and other electrical components. The first and second inflatable chambers 404A, 404B are pressurized to provide the two opposing fluid pressures for fixing the lever 410 at a position corresponding to an angle of the first element with respect to the second element.

In one embodiment, the pressurized fluid is supplied to the first and second inflatable chambers 404A, 404B using a tube 107, such as, a rubber tube, a PE tube, or a PVC tube. The tube 107 may also be used to create a vacuum to remove fluid from the first and second inflatable chambers 404A, 404B within the rotary actuator 400. The tube 107 may be placed inside the rotary actuator 400 or the leg brace 101. The tube 107 may be attached to the first and second inflatable chambers 404A, 404B using glue, a screw, a clip, or other mechanical anchoring structures or chemical bonding. The outer diameter of the tube 107 may be possible, but preferably be less than 4 mm. The fluid supplied to the first and second inflatable chambers 404A, 404B may be a gas or a liquid, and can be recycled or disposable. For example, compressed gas can be applied by way of a pump, a cylinder, or a compressor. The ankle exoskeleton device 100 further comprises a pressure source and a solenoid valve, wherein the pressure source may include a pressure regulator prior to the control valve including, but not limited to, a solenoid or proportional valve to maintain consistent performance. The pressure source may supply pressurized fluid at a first pressure to the first inflatable chamber 404A and a second pressure to the second inflatable chamber 404B. The solenoid valve is configured to control the first pressure and the second pressure supplied to the first and second inflatable chambers 404A, 404B of the rotary actuator 400. Specific examples of gasses include compressed carbon dioxide, air, and nitrogen. These gases may vent to surroundings or be recycled upon depressurization of the first and second inflatable chambers 404A, 404B. The gas can also be drawn out of the first and second inflatable chambers 404A, 404B, creating a vacuum using, for example, a plunger, syringe, or vacuum pump. Examples of liquids include, but are not limited to, water, hydraulic fluid, and mineral oil. As the ankle exoskeleton device 100 is pneumatically driven by a fluid source, this allows the rotary actuator 400 to be lightweight and compliant with human tissue.

In certain embodiments, each of the first and second elastomeric structures 403A, 403B comprises undulated peripheral surfaces defining an inner sinusoidal edge 413 and an outer sinusoidal edge 412. The inner sinusoidal edge 413 and the outer sinusoidal edge 412 allow compression or expansion of the first and second elastomeric structures 403A, 403B based on the vertical force exerted thereto by the first and second inflatable chambers 404A, 404B.

The first and second elastomeric structures 403A, 403B comprise a structural mesh 405 connecting the inner sinusoidal edge 413 and the outer sinusoidal edge 412 for supporting the first and second elastomeric structures 403A, 403B and restricting an expansion of the first and second elastomeric structures 403A, 403B in the vertical direction. The structural mesh 405 is formed using supporting lengthwise filaments, supporting widthwise filaments, or a patterned mesh selected from a net mesh or a double helical mesh. In one embodiment, the structural mesh 405 is impregnated within the first and second elastomeric structures 403A, 403B or mounted to a surface of the first and second elastomeric structures 403A, 403B. The structural mesh 405 connects inner peaks 413A of the inner sinusoidal edge 413 with outer peaks 412A of the outer sinusoidal edge 412, and inner valleys 413B of the inner sinusoidal edge 413 with outer valleys 412B of the outer sinusoidal edge 412. The structural mesh 405 can be made of any metals, for example, titanium alloy, stainless steel, or iron, that are hard to be broken by the fluid pressure inside the first and second inflatable chambers 404A, 404B due to expansion.

In one example, if a smaller pressure is developed in the first inflatable chamber 404A than in the second inflatable chamber 404B, a smaller vertical force would be applied to the first elastomeric structure 403A. Therefore, the lateral force pushing the lever 410 in an anticlockwise direction is greater than that in a clockwise direction. The rotary actuator 400 is driven to cause rotation 408 of the insole 102 by an angle for supporting the ankle joint 50 in plantar flexion 301. This is conceptually illustrated in FIG. 4B. In another example, if greater pressure is developed in the first inflatable chamber 404A than in the second inflatable chamber 404B, a greater vertical force would be applied to the first elastomeric structure 403A. Therefore, the lateral force pushing the lever 410 in a clockwise direction is greater than that in an anticlockwise direction. The rotary actuator 400 is driven to cause rotation 408 of the insole 102 by an angle for supporting the ankle joint 50 in dorsiflexion 302. This is conceptually illustrated in FIG. 4C.

As discussed above, the rotary actuator 400 is pneumatically driven and the rotary shaft 402 is adapted to be moved by controlling two opposing fluid pressures inside the rotary actuator 400. Although it is preferred that the rotary actuator 400 is pneumatically driven, it is apparent that the rotary actuator 400 may otherwise be mechanically driven, electromechanically driven, magnetically driven, or electromagnetically driven. If the rotary actuator 400 is electromagnetically driven, the rotary actuator 400 may comprise a servo motor or a stepping motor for controlling the rotary shaft 402.

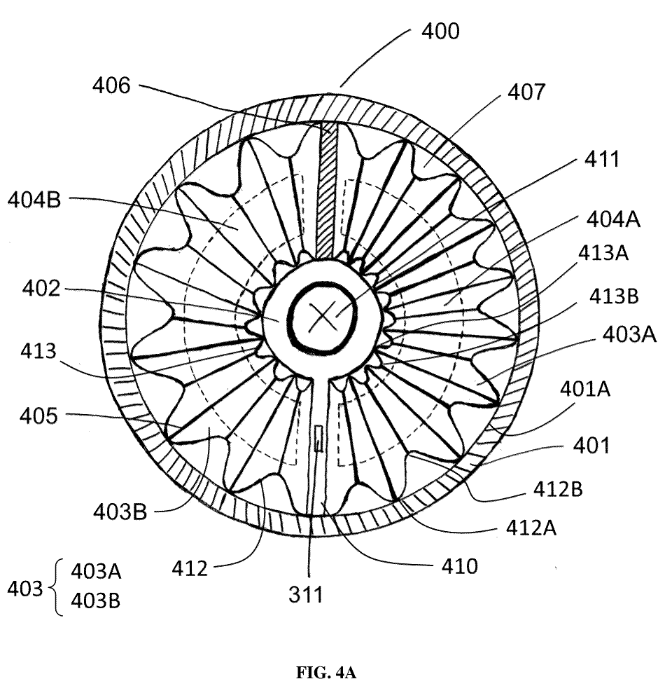

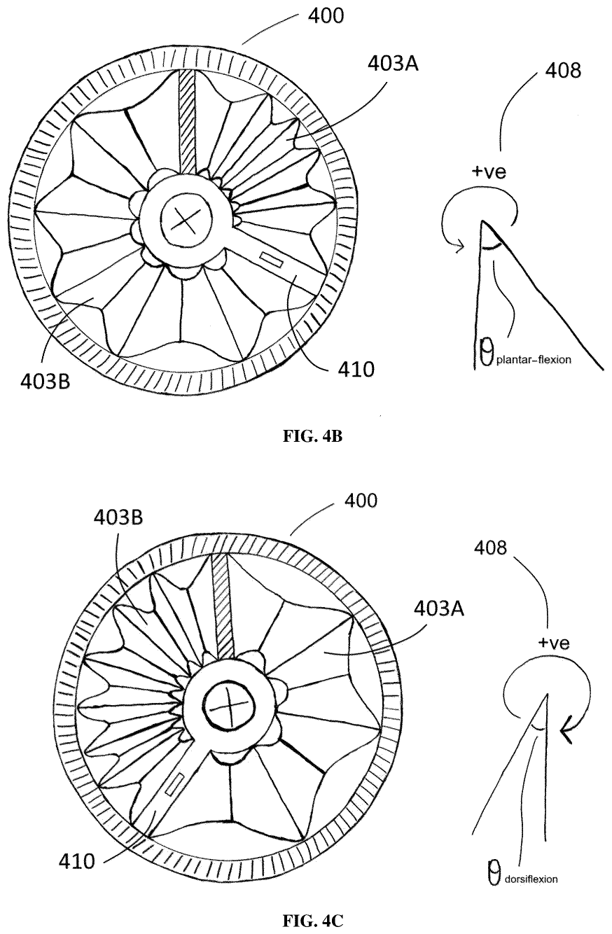

Since the stroke patients suffer from dropped foot 501, the ankle joint 50 at the foot is most likely in a plantar flexion 301 configuration due to weakened muscle coordination and strength after a stroke, as shown in FIG. 5. The exoskeleton ankle 100 provides a torque and range-of-motion to move the ankle joint 50 in a sagittal plane, which is represented by the spatial relationship between the shank 503 and the foot 504.

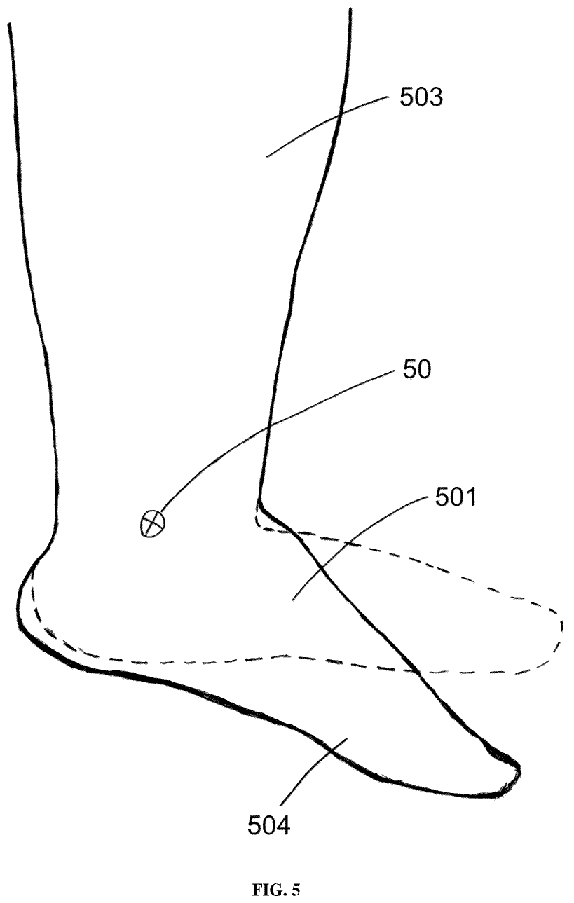

A second embodiment of the present disclosure provides an electrical stimulation system 600 with a plurality of electrodes 610 for applying electrical current pulses 603 to the dorsiflexor muscle group 601 and the plantar flexor muscle group 602, as illustrated in FIG. 7. A plurality of electrodes 610 are arranged for contacting a plurality of regions of the lower limb 10. In certain embodiments, two of the plurality of electrodes 610 are arranged to stimulate the dorsiflexor muscle group 601, including but not limited to the tibialis anterior muscle. Another two of the plurality of electrodes 610 are arranged to stimulate the plantar flexor muscle group 602, including but not limited to the soleus, gastrocnemius, and plantaris. Electrical current pulse 603 can be applied to the dorsiflexor muscle group 601 and the plantar flexor muscle group 602 through an electrical stimulation system 600 to facilitate a better gait.

FIG. 7 shows a schematic diagram illustrating the functioning of electrical stimulation system 600. The input signals from a characteristic control module 700 of the electrical current pulse 603 are determined. The characteristic control module 700 is a circuit module for determining the required frequency, period, pulse width, amplitude, etc., and providing an input signal to the pulse generator 701 that generates the electrical current pulse 603. In certain embodiments, the characteristic is predetermined and optimized. In other alternative embodiments, the characteristic is adjustable by the user using a smartphone or a computer device to meet the need of each user. The characteristic control module 700 may comprise a timer (for example, IC555), a signal generator, or a computer program (for example, LabView, MATLAB, Python, and other application programs). An optocoupler 702, for example, 4N35, is inserted between the pulse generator 701 and the characteristic control module 700 for ground isolation.

In certain embodiments, the pulse generator 701 is configured to generate the electrical current pulses 603 of a predetermined current amplitude independent of a voltage level and a human skin resistance across any two of the plurality of electrodes 610, for example, across the dorsiflexor muscle group 601 and the plantar flexor muscle group 602. In certain embodiments, the pulse generator 701 is a closed-loop system comprising a current-controlled source 704 (for example, TIP 120, TIP 122, 2N4441, etc.) and a transformer 703. The transformer 703 is attached to a current-controlled source 704 at the side of the primary coil for maintaining a constant amplitude of the electrical current pulse 603. The secondary coil of the transformer 703 is connected to the plurality of electrodes 610 that are placed on a muscle for applying electrical current pulses 603 to said muscle. Feedback is obtained from the plurality of electrodes 610. In certain embodiments, the closed-loop system further comprises a voltage buffer 705, a first non-inverting amplifier 706, a differential amplifier 707, and a second non-inverting amplifier 708. The voltage buffer 705 is configured to sense a feedback signal from the plurality of electrodes 610. The feedback signal is then amplified by the first non-inverting amplifier 706, which is connected to the voltage buffer 705. Preferably, the first non-inverting amplifier 706 has a gain equal to or larger than 1. The feedback is further compared with the input signals from the characteristic control module 700. Difference between the feedback signal from the first non-inverting amplifier 706 and the input signal from the characteristic control module 700 is calculated by the differential amplifier 707 and amplified by the second non-inverting amplifier 708. Preferably, the second non-inverting amplifier 708 has a gain equal to or larger than 6. The current-controlled source 704 receives signals from the second non-inverting amplifier 708 to stabilize the amplitude of the electrical current pulse 603, such that the voltage level and the human skin resistance across any two of the plurality of electrodes 610 would not affect the pulse. In certain embodiments, the predetermined current amplitude must not be over 100 mA, which is considered as lethal current and may cause an electric shock to stroke patients. The current pulse 603 may be a square or triangular wave. The electrical stimulation system 600 must comply with electrical safety tests of at least IEC 60601-1-2 (“Medical electrical equipment—Part 1-2: General requirements for basic safety and essential performance—Collateral Standard: Electromagnetic disturbances—Requirements and tests”) and IEC 60601-2-10 (“Medical electrical equipment—Part 2-10: Particular requirements for the basic safety and essential performance of nerve and muscle stimulators”). With the addition of the electrical stimulation system 600, the lower limb can be stimulated during walking to facilitate a better gait. This provides a synergy effect to the rotary actuator 400 as disclosed in the first embodiment.

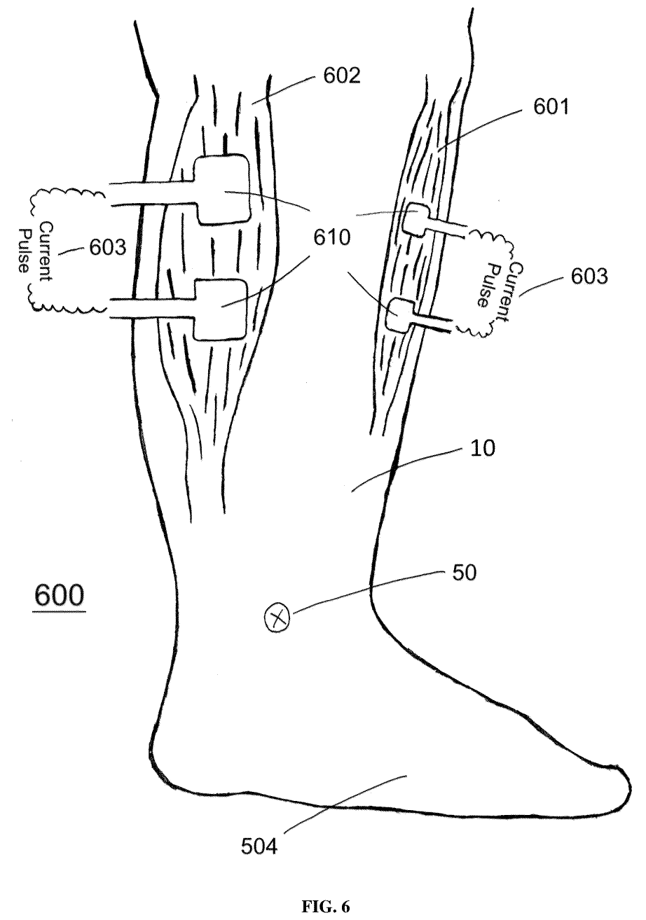

As discussed above, the ankle exoskeleton device 100 comprises a first motion sensor 311 and a second motion sensor 312 placed at one or more locations, including the leg brace 101, the insole 102, or the rotary shaft 402. With the first and second motion sensors 311, 312, the angular and linear displacement, velocity, and acceleration of the dropped foot 501 can be monitored. In certain embodiments, the motion sensors 311, 312 include, but are not limited to, an inertial measurement unit (IMU), an angle encoder, a potentiometer, a strain gauge, a gyroscope, or a flex sensor. Readings from the motion sensors 311, 312 can be analyzed and presented as a spatial gait parameter including, but not limited to, a step width 800, a step length 801, a foot angle 802, a stride length 803, a trajectory of human center-of-mass 804, and a walking speed 805 to quantify the performance of walking of the user, as seen in FIG. 8. Furthermore, the spatial relationship between a shank 503 and the foot 504, for example, ankle configuration (dorsiflexion or plantar-flexion), ankle range-of-motion, foot titling, and ankle spasticity, can be monitored by the ankle exoskeleton device 100 according to an embodiment of the present invention.

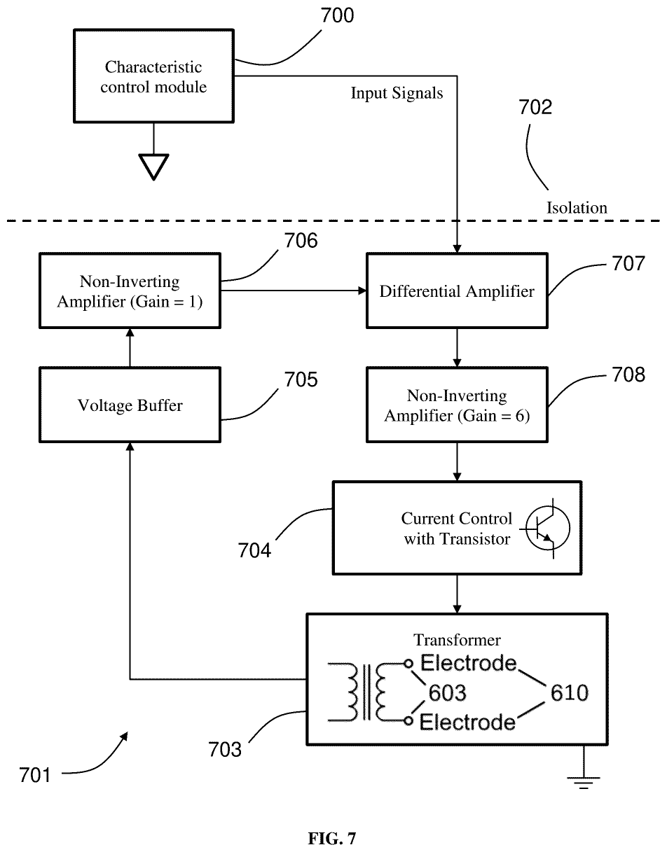

In certain embodiments, at least two force sensors 106 are placed at the heel and forefoot of the insole 102 to measure the stepping force at the heel and toe, respectively. The two force sensors 106 are configured to sense the foot loading at the two portions of the foot, which can render heel ON/OFF and toc ON/OFF respectively by exceeding an activation force threshold, as shown in FIG. 9. This can also be a standing calibration prior to the operation of the ankle exoskeleton device 100. An example of the standing calibration includes, but is not limited to, requiring the user to stand for at least 10 seconds on the floor. The measured values during the calibration period are averaged and defined as a baseline 900 for the sensors. An ON state would be indicated by the force sensor 106 if a loading detected around the area of the heel or toe is greater than the baseline value 900, or vice versa for an OFF state.

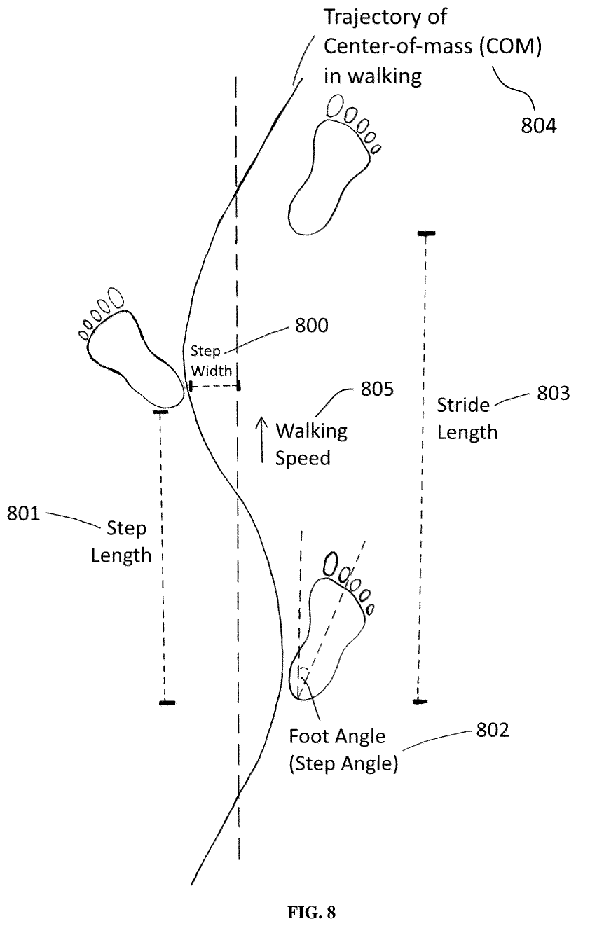

The ON/OFF states of both the heel and toe as detected by the two force sensors 106 may determine the corresponding actions of the ankle exoskeleton device 100 according to an embodiment of the present disclosure. A normal gait pattern of human is shown in FIG. 10. The assisted movement and stimulated muscles by the ankle exoskeleton device 100 may depend on the on-going gait event. Advantageously, the ankle exoskeleton device 100 of the present embodiment provides that the electrical stimulation system 600 is configured to stimulate the dorsiflexor muscle group 601, and the rotary actuator 400 is configured to support the ankle joint 50 to move in dorsiflexion during the gait events of toe-off, mid-swing, terminal swing, and heel strike; and the electrical stimulation system 600 is configured to stimulate the plantar flexor muscle group 602, and the rotary actuator 400 is configured to support the ankle joint 50 to move in plantar flexion during the gait events of terminal stance and pre-swing. For the gait event of loading response and mid-stance, no muscles would be stimulated and no assistance would be provided to any joint movement. The ankle exoskeleton device 100 may be in free motion in this beginning phase.

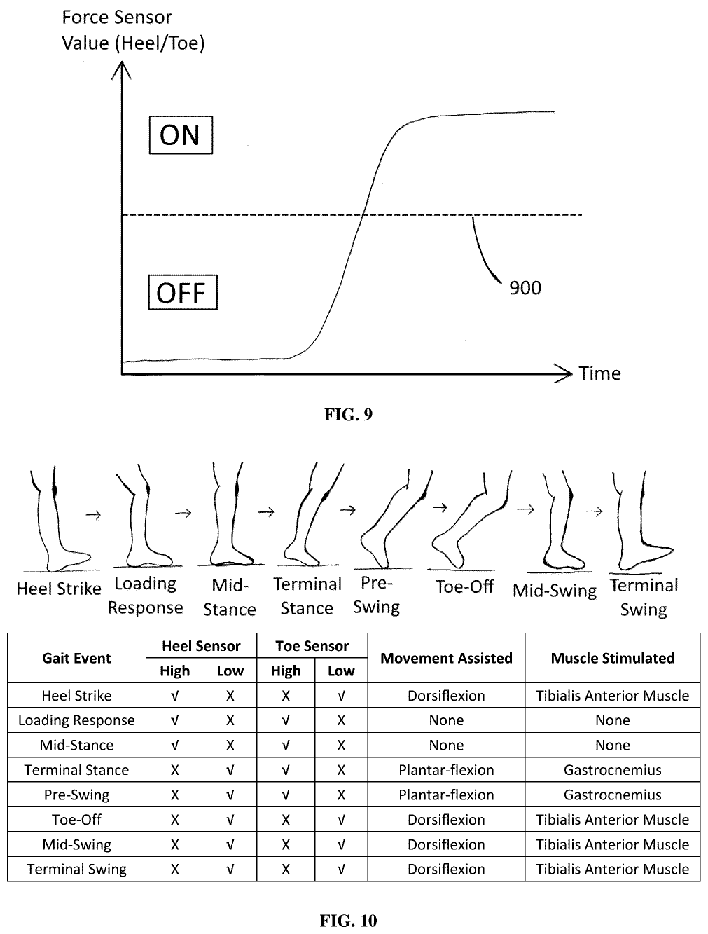

FIG. 11 shows a flowchart 1100 illustrating the operation mechanism of the ankle exoskeleton device 100. The pressure source for the pressurization of the ankle exoskeleton device 100 can be a pump, or any disposable or non-disposable compressed medium including, for example, a carbon dioxide bottle, oxygen tank, compressed nitrogen, or compressed air. A pressure regulator can be provided to maintain constant pressure coming from the pressure source. The pressure source and the pressure regulator may be connected to the first and second inflatable chambers 404A, 404B through a solenoid valve 1102 (or proportional valve) for adjusting the fluid pressure inside the first and second inflatable chambers 404A, 404B by a microcontroller 1101. In certain embodiments, the microcontroller 1101 may be implemented using one or more of: CPU, MCU, controllers, logic circuits, Arduino, Raspberry Pi chip, ATmega, Intel 8051, other digital signal processors (DSP), application-specific integrated circuit (ASIC), Field-Programmable Gate Array (FPGA), or any other digital or analog circuitry configured to interpret and/or to execute program instructions and/or to process information and/or data. The solenoid valve 1102, the electrical stimulation system 600, and the microcontroller 1101 can be placed inside the rotary actuator 400 or the leg brace 101. The pressure source or the pressure regulator may be carried separately by the user or be mounted on any part of the ankle exoskeleton device 100. The microcontroller 1101 is configured to process the readings obtained from the sensor system, and control the activation time of the solenoid valve 1102, thereby the rotary actuator 400 can be controlled to cause rotation of the insole 102 with respect to the leg brace 101. The microcontroller 1101 can also control the electrical stimulation system 600 to activate electrical stimulation on the muscles of the lower limb 10.

When the readings from the two force sensors 106 at the heel and toe are both greater than the baseline 900 (Heel HIGH, Toe HIGH 1111), the microcontroller 1101 will turn off the solenoid valve 1102 and the fluid pressure inside the first and second inflatable chambers 404A, 404B is released to the atmosphere. At the same time, the microcontroller 1101 will turn off the electrical stimulation system 600 and electrical current pulse 603 would not be applied to the muscles. Such no actuation and no stimulation state is the Free Motion state 1104. When at least one of the readings from the two force sensors 106 is less than the baseline 900, the microcontroller 1101 will turn on the solenoid valve 1102 and the fluid pressure inside the first and second inflatable chambers 404A, 404B is increased for actuating the rotary actuator 400. In this case, if the reading from the second force sensor 106B is greater than the baseline 900 (Heel LOW, Toe HIGH 1112), the ankle plantar-flexion 1105 would be supported and the gastrocnemius would be stimulated. Otherwise, the ankle dorsiflexion 1103 would be supported and the tibialis anterior muscle would be stimulated.

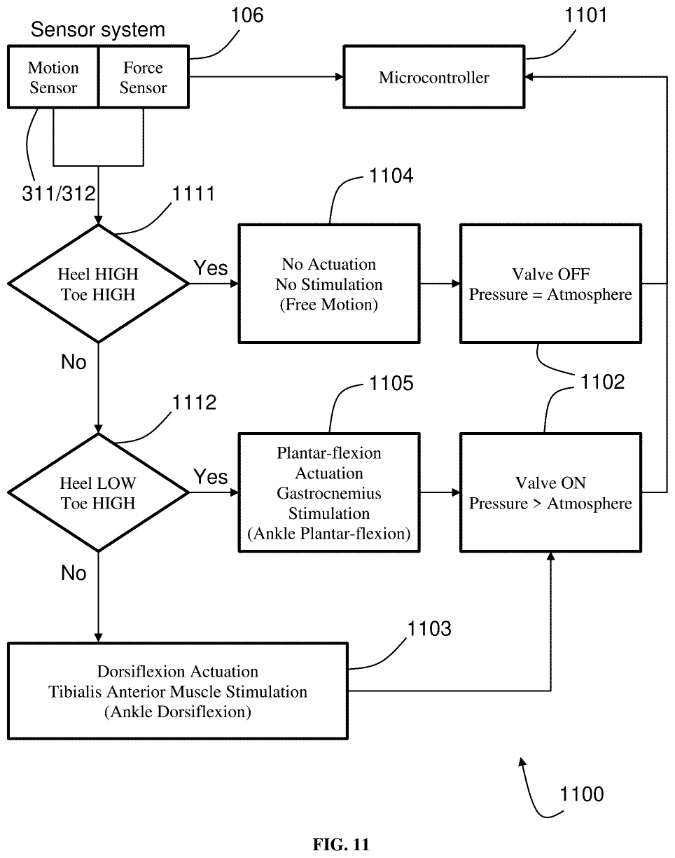

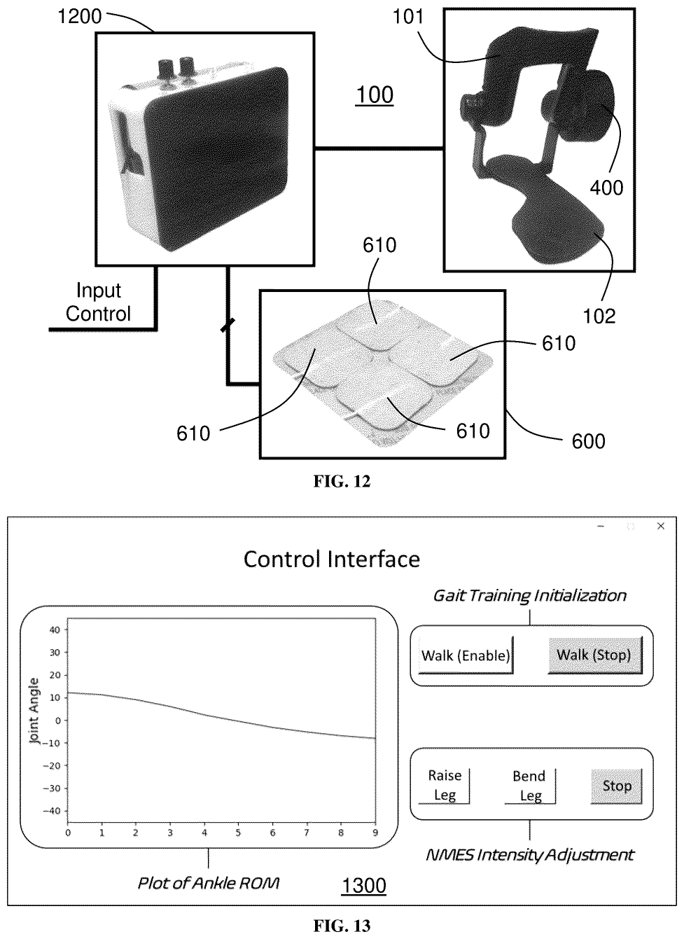

FIG. 12 shows a block diagram of an exemplary ankle exoskeleton device 100 in accordance with certain embodiments of the present disclosure. A control device 1200 is provided for controlling the rotary actuator 400 and the plurality of electrodes 610 of the electrical stimulation system 600. The control device 1200 may comprise one or more components selected from the group consisting of the microcontroller 1101, the pressure source, the pressure regulator, the solenoid valve 1102, a communication device, and a battery receptacle for receiving a power source for powering the ankle exoskeleton device 100. The battery receptacle may be arranged to receive one or more batteries, such as but not limited to one or more replaceable lithium batteries. Input control signals may be provided to the control device 1200 using an input device or via the communication device. The input device may include one or more of: keyboard, mouse, stylus, image scanner (e.g., barcode identifier and QR code), microphone, touch-sensitive screen, image/video input device (e.g., camera device), biometric data input device (e.g., fingerprint detector, facial detector), and other sensors. For the case of using a communication device, the user may control the ankle exoskeleton device 100 using a control interface 1300 of FIG. 13. A person skilled in the art would appreciate that the control device 1200 described above is merely exemplary and that the control device 1200 may have different configurations (e.g., additional components, fewer components, etc.).

The communication device may include one or more of: a modem, a Network Interface Card (NIC), an integrated network interface, an NFC transceiver, a ZigBee transceiver, a Wi-Fi transceiver, a Bluetooth® transceiver, a radio frequency transceiver, an optical port, an infrared port, a USB connection, or other wired or wireless communication interfaces. The transceiver may be implemented by one or more devices (integrated transmitter(s) and receiver(s), separate transmitter(s) and receiver(s), etc.). The communication link(s) may be wired or wireless for communicating commands, instructions, information, and/or data.

The exemplary control interface 1300 is preferably operable using a computer, a smartphone, a tablet, or other computer devices. The control interface 1300 allows the user to control the ankle exoskeleton device 100. In one embodiment, the user can monitor the readings from the sensor system, control the gait training, and set the intensity of the electrical current pulse 603.

It will also be appreciated that where the systems of the present disclosure are either wholly implemented by a computing system or partly implemented by computing systems then any appropriate computing system architecture may be utilized. This will include stand-alone computers, network computers, and dedicated or non-dedicated hardware devices.

This illustrates the fundamental structure of the ankle exoskeleton device 100 in accordance with the present disclosure. It will be apparent that variants of the above-disclosed and other features and functions, or alternatives thereof, may be combined into many other different kinds of assistive devices. The present embodiment is, therefore, to be considered in all respects as illustrative and not restrictive. The scope of the disclosure is indicated by the appended claims rather than by the preceding description, and all changes that come within the meaning and range of equivalency of the claims are therefore intended to be embraced therein.

What is claimed is:

1. A rotary actuator for use in a robotic device, the rotary actuator being pneumatically driven to cause rotation of a first element with respect to a second element about a central axis, the rotary actuator comprising:
a first elastomeric structure;
a second elastomeric structure;
a rotary shaft rotatable about the central axis to generate an output torque for producing a relative rotatory movement of the first element; and
a lever protruding from the rotary shaft perpendicularly for rotating the rotary shaft,
wherein:
the rotary shaft is adapted to be moved by controlling two opposing fluid pressures inside the rotary actuator; and
the first elastomeric structure is extendable to push the lever in a clockwise direction, and the second elastomeric structure is extendable to push the lever in an anticlockwise direction, thereby the first and second elastomeric structures are arranged to control the rotary shaft.

2. The rotary actuator of claim 1, wherein:
the rotary actuator is coupled to the first element and the second element, wherein the first element comprises an insole, and the second element comprises a leg brace;
the insole is pivotally coupled to the leg brace at or proximal to an ankle joint; and
the insole is movable by the rotary actuator with a range of motion to support the ankle joint to move between plantar flexion and dorsiflexion in a sagittal plane.

3. The rotary actuator of claim 1 further comprising a first inflatable chamber and a second inflatable chamber, wherein:
the first inflatable chamber is in contact with the first elastomeric structure, and the second inflatable chamber is in contact with the second elastomeric structure; and
the first and second inflatable chambers are pressurized to provide the two opposing fluid pressures for fixing the lever at a position corresponding to an angle of the first element with respect to the second element.

4. The rotary actuator of claim 3, wherein the first and second inflatable chambers are pressurized to provide the two opposing fluid pressures for fixing the lever at the position corresponding to the angle of the first element with respect to the second element.

5. The rotary actuator of claim 3, wherein the rotary actuator is provided within a hollow cylinder defining an internal space between an inner wall of the hollow cylinder and the rotary shaft, and wherein the first and second elastomeric structures and the first and second inflatable chambers are accommodated within the internal space.

6. The rotary actuator of claim 3, wherein the first inflatable chamber receives pressurized fluid at a first pressure from a pressure source; and the second inflatable chamber receive pressurized fluid at a second pressure from the pressure source.

7. The rotary actuator of claim 6, wherein the first pressure and the second pressure supplied to the first and second inflatable chambers are controlled by a control valve.

8. The rotary actuator of claim 1, wherein each of the first and second elastomeric structures comprises undulated peripheral surfaces defining an inner sinusoidal edge and an outer sinusoidal edge; wherein the inner and outer sinusoidal edges allow compression or expansion of the first and second elastomeric structures.

9. The rotary actuator of claim 8, wherein the first and second elastomeric structures comprise a structural mesh connecting the inner sinusoidal edge and the outer sinusoidal edge for supporting the first and second elastomeric structures and restricting an expansion of the first and second elastomeric structures in a vertical direction.

10. The rotary actuator of claim 9, wherein the structural mesh connects inner peaks of the inner sinusoidal edge with outer peaks of the outer sinusoidal edge; and inner valleys of the inner sinusoidal edge with outer valleys of the outer sinusoidal edge.

11. The rotary actuator of claim 9, wherein the structural mesh is formed using supporting lengthwise filaments, supporting widthwise filaments, or a patterned mesh selected from a net mesh or a double helical mesh.

12. The rotary actuator of claim 9, wherein the structural mesh is impregnated within the first and second elastomeric structures or mounted to a surface of the first and second elastomeric structures.

13. An ankle exoskeleton device, comprising:
a leg brace;
an insole pivotally coupled to the leg brace at or proximal to an ankle joint;
a rotary actuator coupled to the leg brace and the insole, the rotary actuator being driven to cause rotation of the insole with respect to the leg brace to support the ankle joint; and
an electrical stimulation system comprising a pulse generator and a plurality of electrodes for intermittently stimulating muscles in a lower limb to facilitate a walking gait,
wherein:
the plurality of electrodes are arranged for contacting a plurality of regions of the lower limb to apply electrical current pulses to dorsiflexor muscle group and plantar flexor muscle group; and
the pulse generator is configured to generate the electrical current pulses of a predetermined current amplitude independent of a voltage level and a human skin resistance across any two of the plurality of electrodes.

14. The ankle exoskeleton device of claim 13, wherein the pulse generator is a closed-loop system comprising a current-controlled source and a transformer having a primary coil connected to the current-controlled source and a secondary coil connected to the plurality of electrodes.

15. The ankle exoskeleton device of claim 14, wherein the closed-loop system further comprising:
a voltage buffer configured to sense a feedback signal from the plurality of electrodes;
a first non-inverting amplifier connected to the voltage buffer to amplify the feedback signal with a gain equal to or larger than 1; and
a differential amplifier configured to determine a difference between the feedback signal from the first non-inverting amplifier and an input signal from a characteristic control module.

16. The ankle exoskeleton device of claim 13, wherein the electrical current pulses have a square waveform or a triangular waveform.

17. The ankle exoskeleton device of claim 13, wherein two of the plurality of electrodes are arranged to stimulate the dorsiflexor muscle group, including tibialis anterior muscle; and another two of the plurality of electrodes are arranged to stimulate the plantar flexor muscle group, including soleus, gastrocnemius, and plantaris.

18. The ankle exoskeleton device of claim 17, wherein:
the electrical stimulation system is configured to stimulate the dorsiflexor muscle group, and the rotary actuator is configured to support the ankle joint to move in dorsiflexion during toe-off, mid-swing, terminal swing, and heel strike; and
the electrical stimulation system is configured to stimulate the plantar flexor muscle group, and the rotary actuator is configured to support the ankle joint to move in the plantar flexion during terminal stance and pre-swing.

19. The ankle exoskeleton device of claim 13 further comprising a sensor system for providing kinetic feedback and kinematic feedback, wherein the sensor system further comprises:
a first motion sensor placed on the leg brace to sense a change in displacement and orientation of the leg brace;
a second motion sensor placed inside the rotary actuator to sense a change in displacement and orientation of the insole; and
at least two force sensors placed on the insole at a heel region and a toe region.

20. The ankle exoskeleton device of claim 19 further comprising a microcontroller configured to process the readings obtained from the sensor system; control the rotary actuator to cause rotation of the insole with respect to the leg brace; and control the electrical stimulation system to activate electrical stimulation on muscles of the lower limb.
